(19) United States
(12) Patent Application Publication
(10) Pub. No.: US 2009/0131512 A1
(43) Pub. Date: May 21, 2009

(54) Inhibition of type I in IFN production

(75) Inventors: Franck Barrat, San Francisco, CA (US); Cristiana Guiducci, Albany, CA (US); Vassili Soumelis, Paris (FR)

Correspondence Address: ROBINS & PASTERNAK, 1731 EMBARCADERO ROAD, SUITE 230, PALO ALTO, CA, 94303, US

(73) Assignees: Dynavax Technologies Corp.; Institut Curie; INSERM

(21) Appl. No.: 12/290,654

(22) Filed: Oct. 30, 2008

Related U.S. Application Data
(60) Provisional application No. 61/001,093, filed on Oct. 31, 2007.
(60) Provisional application No. 61/010,674, filed on Jan. 10, 2008.

(51) Int. Cl. A61K 31/366; A61P 37/00

(52) U.S. Cl. 514/453

(57) ABSTRACT

The invention provides methods for decreasing type I IFN production by human plasmacytoid dendritic cells in response to TLR activation.

### CROSS-REFERENCE TO RELATED APPLICATIONS

The present application claims the benefit of U.S. Provisional Application Nos. 61/001,093, filed Oct. 31, 2007 and 61/010,674, filed Jan. 10, 2008; the disclosures of which are hereby incorporated by reference in their entireties herein.

### STATEMENT OF RIGHTS TO INVENTIONS MADE UNDER FEDERALLY SPONSORED RESEARCH

Not applicable.

### TECHNICAL FIELD

The present invention relates to methods of inhibiting type I IFN production and to treatment, prevention and/or delay of diseases caused by overproduction of type I IFN. The invention also relates to amelioration of symptoms associated with pathogenic type IFN.

### BACKGROUND

Type I interferon (IFN) are known to be involved in a variety of immune responses. Plasmacytoid dendritic cell (DC) precursors (pDC) are the main type I interferon (IFN) producers in human and mouse (Liu (2005) Annu. Rev. Immunol. 23:275-306). PDC play a key role in innate anti-viral immune responses but can also evolve into potent antigen presenting cells and be important players in adaptive responses (Liu (2005) Annu. Rev. Immunol. 23:275-306; Colonna et al. (2004) Nat. Immunol. 5:1219-1226). Activation of pDC through TLR7 and TLR9 can trigger both innate and adaptive immune responses, including production of large quantities of type I IFN production and/or DC differentiation (Liu (2005) Annu. Rev. Immunol. 23:275-306). For example, synthetic CpG-containing oligonucleotides of the types A and B (CpG-A, CpG-B) selectively induce type I IFN production and DC differentiation, respectively while some microbial stimuli, such as influenza virus (Flu), herpes simplex virus (HSV) or CpG-C can induce simultaneously both responses (Duramad et al. (2003) Blood 102:4487-4492).

Two factors seem to be important for the induction of large quantities of type I IFN in pDC: (i) the ability of the TLR ligand to bind its receptor in the early endosomal compartments (Honda et al. (2005) Nature 434:1035-1040; Guiducci et al. (2006) J. Exp. Med. 203:1999-2008); and (ii) the phosphorylation and nuclear translocation of the transcription factor IRF-7 (Honda et al. (2005) Nature 434:772-777). Nuclear translocation of the transcription factor IRF-7 has been shown to depend on the kinases IRAK-1 (Uematus et al. (2005) J. Exp. Med. 201:915-923) and IkB kinase-α (IKK-α) (Hoshino et al. (2006) Nature 440:949-953) in mouse pDC.

The phosphatidylinositol-3 kinase (PI3K) pathway is involved in a variety of biological processes, including cell survival and proliferation, B and T cell receptor signaling, as well as activation of G-protein-coupled receptors, such as chemokine receptors (Deane et al. (2004) Annu. Rev. Immunol. 22:563-598). PI3K contains regulatory subunits (p85 α, β) and catalytic subunits (p110 α, β, γ and δ). PI3K γ and δ are preferentially expressed in cells of hemopoietic origin, whereas expression PI3K α and β expression is ubiquitous. Accordingly, knockout mice for p110 α and β show embryonic lethality while knockout mice for p110 γ and δ are viable and fertile and show altered phenotype exclusively when their immune system is under acute stress (Rommel et al. (2007) Nat. Rev. Immunol. 7:191-201).

Furthermore, the PI3K pathway has been shown to be activated by various TLR-ligands, including unmethylated CpGs (Ishii et al. (2002) J. Exp. Med. 196:269-274), and can function as a positive or negative regulator of TLR responses depending on the cell type and the TLR ligand used (Fukao et al. (2003) Trends Immunol. 24:358-363). Inhibition of PI3K in mouse myeloid DC and macrophages increased IL-12 production in response to TLR stimulation (Fukao et al. (2003)), a result compatible with in vivo observation of a skewed Th1 response in PI3K p85α^{-/-} mice ((Fukao et al. (2002) Nat. Immunol. 3:875-881)) and susceptibility to microbial induced sepsis in mice through an increased production of innate cytokines (Williams et al. (2004) J. Immunol. 172:449-456).

In murine CD4+ T cells, MyD88 was recently shown to activate PI3K and to enable CpG-mediated proliferation, but did not effect survival (Gelman et al. (2006) Immunity 25:783-793). In murine macrophages, however, CpG ODN promoted survival through TLR-9 and the PI3K pathway (Sester et al. (2006) J. Immunol. 177:4473-4480).

However, the role of PI3K in human pDC is not known and the molecular mechanism and cell type specificity of PI3K has not been determined. Differences in the role of PI3K in cell lines as compared to primary cells have been reported (Deane et al. (2004) Annu. Rev. Immunol. 22:563-598). Furthermore, in mouse pDC, the PI3K inhibitor wortmannin inhibited autophagy in Flu-activated mouse pDC without any effect in type I IFN production (Lee et al. (2007) Science 315:1398-1401).

There remains a need for strategies of controlling type I IFN production by regulating PIK3.

All patents, patent applications, and publications cited herein are hereby incorporated by reference in their entirety.

### SUMMARY

The invention relates to inhibition of type I IFN production in primary cells, preferably human pDC, using PI3K inhibitors, preferably specific PI3K subunit (delta) inhibitors. The invention also relates to methods of treating, preventing and/or delaying the onset of any disease caused by overproduction of type I IFN as well as to methods of treating symptoms associated with pathogenic type I IFN.

In one aspect, the invention provides a method for regulating type I IFN production in a primary cell, preferably a human pDC, by administering a composition comprising a PI3K inhibitor. In certain embodiments, the PI3K inhibitor is specific for the delta (δ) subunit of PI3K. The compositions may also include, for example, a pharmaceutically acceptable excipient or any of a number of other components.

In another aspect, the invention provides methods of regulating IRF-7 nuclear transport in human pDC by administering a composition comprising a PI3K inhibitor. In certain embodiments, the PI3K inhibitor is specific for PI3K δ subunit. The compositions may also include, for example, a pharmaceutically acceptable excipient or any of a number of other components.

In another aspect, the invention provides methods of inhibiting a TLR stimulated type I IFN production response in an individual, comprising administering to an individual an inhibitor of PI3K (e.g., an inhibitor specific for PI3K δ subunit) or a composition comprising the inhibitor in an amount sufficient to suppress TLR (e.g. TLR7/9) dependent type I IFN production in said individual.

In another aspect, the invention provides methods of treating an individual with a disease caused or characterized by the presence of pathogenic type I IFN, comprising administering to the individual a composition comprising a PI3K inhibitor in an amount sufficient to inhibit pathogenic type I IFN production in said individual. In certain embodiments, the PI3K inhibitor is specific for the delta (δ) subunit of PI3K. The disease characterized or caused by increased production of type I IFN may be, for example, an autoimmune disease such as systemic lupus erythematosus (SLE), rheumatoid arthritis, psoriasis or Sjögren's disease.

In another aspect, the invention provides methods of ameliorating one or more symptoms associated with overproduction of type I IFN, comprising administering an effective amount of a PI3K δ subunit inhibitor to an individual experiencing the symptoms. Administration of a PI3K δ subunit inhibitor ameliorates one or more symptoms, for example may ameliorate the symptoms of an autoimmune disease, including SLE, rheumatoid arthritis, psoriasis or Sjögren's disease.

In another aspect, the invention provides methods of preventing or delaying development of a disease characterized or caused by overproduction of type I IFN, comprising administering an effective amount of a PI3K δ subunit inhibitor to an individual at risk of developing the disease. Administration of a PI3K δ subunit inhibitor prevents or delays development of the disease. In certain embodiments, the disease is an autoimmune disease.

In any of the methods described herein, the PI3K inhibitor may inhibit a TLR9 dependent cell response, a TLR7/8 dependent cell response, and/or a TLR7/8/9 dependent cell response.

The invention further relates to kits, preferably for carrying out the methods of the invention. The kits of the invention generally comprise a PI3K δ subunit inhibitor (generally in a suitable container), and may further include instructions for use of the inhibitor in regulation of type I IFN of an individual.

The invention further concerns the use of a PI3K inhibitor, in particular an inhibitor specific for PI3K δ subunit, for the preparation of a medicament for regulating type I IFN production. In particular, the medicament is designed to inhibit a TLR stimulated type I IFN production response. In a preferred embodiment, the invention concerns the use of a PI3K inhibitor, in particular an inhibitor specific for PI3K δ subunit, for the preparation of a medicament for treating a disease caused or characterized by the presence of pathogenic type I IFN, for ameliorating one or more symptoms associated with overproduction of type I IFN or for preventing or delaying development of a disease characterized or caused by overproduction of type I IFN, preferably an autoimmune disease including SLE, rheumatoid arthritis, psoriasis or Sjögren's disease, or a symptom thereof.

The invention also concerns a PI3K inhibitor, in particular an inhibitor specific for PI3K δ subunit, for regulating type I IFN production, in particular for inhibiting a TLR stimulated type I IFN production response. In a preferred embodiment, the invention concerns a PI3K inhibitor, in particular an inhibitor specific for PI3K 6 subunit, for treating a disease caused or characterized by the presence of pathogenic type I IFN, for ameliorating one or more symptoms associated with overproduction of type I IFN or for preventing or delaying development of a disease characterized or caused by overproduction of type I IFN, preferably an autoimmune disease including SLE, rheumatoid arthritis, psoriasis or Sjögren's disease, or a symptom thereof.

### BRIEF DESCRIPTION OF THE DRAWINGS

FIG. 1, panels A and B, are histograms showing activation of the PI3K pathway in human PDC by TLR-activating CpGs. Purified PDC were cultured with 1 μM CpG-C (FIG. 1A) or heat-inactivated Influenza virus (Flu) (2 MOI) (FIG. 1B) with or without the PI3K inhibitor LY294002 (LY) at 1 μM and stained with anti-pAKT after 20 minutes (left panels) or 90 minutes (right panels). Representative histograms of at least three separate experiments are shown.

FIG. 2, panels A to C, are graphs showing P13K inhibition of TLR7 and TLR9 mediated IFN-α responses in human PDC. FIG. 2A shows IFN-α levels in purified pDCs cultured for 16 hours with 1 μM CpG-C (left panel), 5 MOI UV-inactivated Herpes Simplex Virus (HSV) (middle panel) or 1 MOI Flu (right panel) either alone or in combination with various concentrations of PI3K inhibitor LY (indicated on the x-axis of each graph in μM). IFN-α production was evaluated by ELISA. Averages of one experiment with 3 independent donors (representative of over 15 donors) are shown. FIG. 2B depict expression levels of IFN-α1 (left panels), IFN-ω (middle panels) and IFN-β (right panels) in purified PDC cultured with CpG-C (1 μM) alone or in the presence of LY inhibitor (2 μM) for 2 hours (top panels) and 5 hours (bottom panels). The expression levels of were measured by real-time PCR. The average of three independent donors is shown. FIG. 2C shows IFN-α production (as evaluated by ELISA) from purified pDC cultured with CpG-C (left panels) or Flu (right panels), either alone or in the presence of LY inhibitor. IFN-α production was evaluated from supernatants collected after 5 hours (top panels) and from cells that were washed twice and restimulated with CpG-C or Flu for another 12 hours (bottom panels). The average of 3 independent donors is shown. Data were analyzed using a 2-tailed Student's t test. Differences were considered significant (*) at a P level less than 0.05.

FIG. 3, panels A to D, show that P13K inhibition does not effect production of inflammatory cytokines or maturation of PDC in response to TLR7/9 triggering. FIG. 3A shows the amounts of IL-6 (top panel) and TNF-α (bottom panel) produced from purified PDC cultured with CpG-C ISS (1 μM) or HSV (5 MOI) either alone or in combination with various concentration of PI3K inhibitor LY (μM administered shown on x-axis) for 16 hr. FIG. 3B shows the amounts of IL-6 (top panel) and TNF-α (bottom panel) produced from purified PDC cultured with Flu (1 MOI) viruses either alone or in combination with various concentration of PI3K inhibitor LY (μM) for 16 hr. IL-6 and TNF-α production was evaluated by ELISA. Averages of one experiment with 3 independent donors (representative of over 15 donors) are shown. FIG. 3C shows expression levels of IL-6 (top panels) and TNF-α (bottom panels) from purified PDC were cultured with CpG-C (1 μM) alone or in the presence of LY inhibitor (2 μM) after 2 hours (left panels) and 5 hours (right panel). The expression levels of IL-6 and TNF-α were measured by real-time PCR and the average of three independent donors is shown. FIG. 3D shows results of cells stimulated as indicated above and characterized for CD80 and CD86 expression by flow cytometry analysis. Data shown are representative of at least 10 donors (see, also FIG. 9).

FIG. 4, panels A and B, are graphs showing PI3Kδ is essential for IFN-α production by PDC in response to TLR stimulation. FIG. 4A shows expression of class I A p110 (α, β, δ) and class I B (γ) PI3K subunits in fresh human pDC (left panel), pDC in medium (middle panel) and pDC cultured with CpG (right panel). Purified pDC were cultured for δ hours as indicated, RNA was extracted and analyzed by quantitative PCR. Expression levels are expressed after normalization to β-actin. Data are shown as mean with standard deviation from three independent donors. FIG. 4B show graphs depicting IFN-α production from purified pDC cultured with CpG-C (1 μM), either alone or in combination with LY and various concentration of PI3K inhibitor p110 γ AS 604850 (right panel) or various concentrations of PI3K inhibitor p110 δ IC 87114 (left panel) for 16 hr. IFN-α production was measured by ELISA. The mean of 3 independent donors is shown.

FIG. 5, panels A to E, show that PI3K is critical for the nuclear translocation of IRF-7 in PDC but does not block IRF-7 upregulation. FIG. 5A is a graph showing IRF-7 expression from purified PDC (1×10⁵) cultured with or without CpG-C ISS (1 μM) alone or in the presence of LY inhibitor (5 μM) 2 hours and 5 hours after stimulation. The average of three real time PCR experiments from independent donors is shown. FIG. 5B shows staining of untreated purified pDCs (top panels), CpG stimulated pDCs (middle panels) and CpG stimulated/LY inhibitor treated pDCs (bottom panels). For each experiment, 2×10⁵ purified PDC were left untreated or stimulated with CpG alone or in the presence of LY inhibitor for 3 h. Cells were visualized using the membrane staining of Class II molecule (FITC) (left most column of panels) while the nucleus was identified using DAPI (3^{rd} column of panels from the left). IRF-7 nuclear translocation was visualized by immunofluorescence with IRF-7 antibody (Alexa 555/red) (2^{nd} column of panels from the left). Representative cells of at least 4 independent donors are shown. Bar represent 5 μm. FIG. 5C is a graph depicting the percentage pDCs that were positive for IRF-7 nuclear staining. Between 50-70 cells from at least 4 different donors were analyzed for IRF7 translocation in the nuclei. Cells were considered positive when at least 20% of the IRF-7 fluorescence was localized in the nucleus. FIG. 5D shows NF-kB detection from purified PDC cultured with CpG-C ISS (1 μM) with or without PI3K inhibitor LY (1 μM or 5 μM) or NF-kB inhibitor (0.5 μM) for 90 min. The left and middle panels show representative histograms. Expression intensity values as mean fluorescent intensity. The average of three experiments is shown *P<0.05 (right panel). FIG. 5E is a graph showing fold increase in the binding activity of NF-kB p50 and p65 family on nuclear extracts. Purified PDC were cultured with CpG-C ISS (1 μM) with or without PI3K inhibitor LY (5 μM) for 4 hours. Data are shown as the fold of increase to unstimulated (mean±SEM) of three separate experiments.

FIG. 6 shows IFN-α and IL-6 production in purified pDC cultured with CpG-A, or CpG-B (0.5 μM), either alone or in combination with various concentration of PI3K inhibitor LY (μM), for 16 hr production. IFN-α and IL-6 levels were evaluated by ELISA. The averages of 9 independent donors are shown. Differences were considered significant (*) at a P level less than 0.05. The top panel shows that PI3K inhibition inhibits CpG-A-mediated IFN-α response in human pDC. As shown in the middle and bottom panels, no significant inhibition was observed in IL-6 production in response to both CpG-A and CpG-B.

FIG. 7 is a graph showing PI3K does not affect the pDC survival in response to TLR9 stimulation. Purified pDC were stimulated with CpG-C ISS (1 μM) either alone or in combination with various concentration of PI3K inhibitor LY. After 20 h, viability was assessed using a flow cytometry based viability assay (LIVE/DEAD Viability/Cytotoxicity Kit from Molecular Probe). Average of δ independent donors is shown.

FIG. 8 includes graphs showing CCL2 (top panels) and IP-10 (bottom panels) production by purified pDC cultured with CpG-C (1 μM) alone or in the presence of LY inhibitor (2 μM) for 2 hours (left panel) and 5 hours (right panel). The expression levels of IP-10 and CCL2 were measured by real-time PCR. The average of three independent donors is shown.

FIG. 9 includes graphs showing CD80 (top panels) and CD86 (bottom panels) expression in purified PDC were stimulated with CpG-C ISS (1 μM) or HSV (5 MOI) (left panels) or FLU (1 MOI) virus (right panels), either alone or in combination with various concentration of the PI3K inhibitor LY. After 16 h, cells were characterized for CD80 and CD86 expression by flow cytometry analysis. Histograms show cumulative mean fluorescence intensities from 4 individual donors, representative of at least 10 donors. There was no statistical difference within the groups.

FIG. 10, panels A to C, show that PI3K inhibition does not affect internalization or endosomal localization of CpG ISS in human PDC. FIG. 10A show representative histograms depicting internalization of the fluorescent ISS in purified PDC as evaluated by flow cytometry. PDC were cultured for 3 h either alone (dashed line) or with 0.5 μM CpG-C/Alexa-488 alone (thin line) or in the presence of LY (thick line, middle panel) or wortmannin inhibitors (5 μM) (thick line, right panel). Surface bound fluorescence was quenched with a solution of 100 μg/ml trypan blue in PBS. FIG. 10B shows confocal microscopy images obtained from intracellular staining of purified PDC with anti-transferrin receptor (TfR) or anti-LAMP1 (LP1) antibodies. PDC were cultured with fluorescent CpG-C alone or in the presence of LY inhibitor (5 μM) for 3 h. Cells were then fixed and stained intracellularly and imaged by confocal microscopy. Images were acquired using a ZEISS LSM 510 META confocal microscope. Bar represent 5 μm. FIG. 10C is a graph showing the percentage of PDC as indicated on the x-axis in which the ODN and either Transferrin receptor (TfR) or LAMP-1 (LP) were colocalized. Between 28-70 cells from at least 3 different donors were analyzed.

### DETAILED DESCRIPTION

We show herein that phosphatidylinositol-3 kinase (PI3K) is activated by TLR stimulation in primary human pDC plasmacytoid pre-dendritic cells (pDC), which cells are the main producers of type I interferon (IFN) in response to Toll-like receptor (TLR) stimulation. Furthermore, using specific inhibitors, we have discovered that PI3K is required for type I IFN production by pDC, both at the transcriptional and protein levels. Importantly, we also show that PI3K is not involved in other proinflammatory of pDC including TNF-α and IL-6 production and dendritic cell differentiation. Our findings are in contrast to previous studies showing inhibition of IFN-α in human pDC using specific inhibitors of TLR (Barrat et al. (2005) J. Exp. Med. 202:1131-1139) or following cross-linking of surface ILT7 (Cao et al. (2006) J. Exp. Med. 203:1399-1405) or BDCA2 (Dzionek et al. (2001) J. Exp. Med. 194:1823-1834) induced a decrease in TNF-α and IL-6 production.

We also found that PDC preferentially expressed the PI3K delta subunit, which was specifically involved in the control of type I IFN production. Although uptake and endosomal trafficking of TLR ligands were not affected in the presence of PI3K inhibitors, there was a dramatic defect in the nuclear translocation of IRF-7, while NF-kB activation was preserved. Thus, PI3K selectively controls type I IFN production by regulating IRF-7 nuclear translocation in human pDC. As such, PI3K inhibitors, particularly specific PI3K δ subunit inhibitors can be used to inhibit pathogenic type I IFN to prevent, treat and/or ameliorate the symptoms of auto-immune diseases.

General Techniques

the practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis. (M. J. Gait, ed., 1984); Animal Cell Culture (R. I. Freshney, ed., 1987); Handbook of Experimental Immunology (D. M. Weir & C.C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); and Methods of Immunological Analysis (R. Masseyeff, W. H. Albert, and N. A. Staines, eds., Weinheim: VCH Verlags gesellschaft mbH, 1993).

### DEFINITIONS

As used herein, the singular form “a”, “an”, and “the” includes plural references unless indicated otherwise. For example, “a” PI3K inhibitor includes one or more such inhibitors.

“Adjuvant” refers to a substance which, when added to an immunogenic agent such as antigen, nonspecifically enhances or potentiates an immune response to the agent in the recipient host upon exposure to the mixture.

An “individual” is a vertebrate, such as avian, and is preferably a mammal, more preferably a human. Mammals include, but are not limited to, humans, primates, farm animals, sport animals, rodents and pets.

An “effective amount” or a “sufficient amount” of a substance is that amount sufficient to effect beneficial or desired results, including clinical results, and, as such, an “effective amount” depends upon the context in which it is being applied. In the context of administering a composition that suppresses type I IFN production, an effective amount of a PI3K inhibitor is an amount sufficient to inhibit or decrease Type I IFN production, for example in response to TLR stimulation. An effective amount can be administered in one or more administrations.

The term “co-administration” as used herein refers to the administration of at least two different substances sufficiently close in time to regulate an immune response. Preferably, co-administration refers to simultaneous administration of at least two different substances.

“Suppression” or “inhibition” of a response or parameter includes decreasing that response or parameter when compared to otherwise same conditions except for a condition or parameter of interest, or alternatively, as compared to another condition. For example, a composition comprising a PI3K inhibitor which suppresses type I IFN production as compared to, for example, type I IFN production in cells without the inhibitor.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder. Especially in the autoimmune disease context, as is well understood by those skilled in the art, palliation may occur upon regulation or reduction of the unwanted immune response. Further, palliation does not necessarily occur by administration of one dose, but often occurs upon administration of a series of doses. Thus, an amount sufficient to palliate a response or disorder may be administered in one or more administrations.

As used herein, the term “comprising” and its cognates are used in their inclusive sense; that is, equivalent to the term “including” and its corresponding cognates.

### Methods of the Invention

The invention provides methods of decreasing type I IFN production in a cell, preferably a human plasmacytoid pre-dendritic cell (pDC), comprising administering to the cell a PI3K inhibitor, preferably an inhibitor specific for the δ subunit of PI3K. The invention also provides methods for treating diseases caused by pathogenic type I IFN as well as methods for ameliorating symptoms associated with pathogenic type I IFN production, including, but not limited to, symptoms associated with autoimmunity.

The PI3K inhibitor is administered in an amount sufficient to regulate Type I IFN production. Any PI3K inhibitor can be used, including small molecules, proteins and/or polynucleotides. Non-limiting examples of general PI3K inhibitors include LY294002 and wortmannin, which are commercially available.

In a preferred embodiment, the PI3K inhibitor is specific for the delta (6) subunit of PI3K as administration of a PI3K inhibitor specific for the delta (6) subunit of PI3K may reduce or eliminate toxicity associated with administration of non-specific PI3K inhibitors. A specific of selective PI3Kδ inhibitor refers to any compound that inhibits the PI3Kδ isozyme more effectively than other isozymes of the PI3K family. Specific PI3Kδ inhibitors are understood to be more selective for PI3Kδ than compounds conventionally and generically designated PI3K inhibitors, e.g., wortmannin or LY294002. Compounds of any type that selectively negatively regulate PI3Kδ expression or activity can be used in the methods of the invention. Thus, the PI3K delta inhibitor may comprise one or more molecules described in U.S. Pat. Nos. 6,518,277; 6,800,620; and/or U.S. Patent Application No. 2005/0261317A1, incorporated by reference in their entireties herein. See, also, Example 2. These molecules include but are not limited to, 3-(2-isopropylphenyl)-5-methyl-2-(9H-purin-6-ylsulfanylmethyl)-3H-q-uinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-ylsulfanylmethyl)-3H-quin-azolin-4-one; 3-(2-fluorophenyl)-5-methyl-2-(9H-purin-6-ylsulfanylmethyl)-3H-quin-azolin-4-one; 3-(2-methoxyphenyl)-5-methyl-2-(9H-purin-y-ylsulfanylmethyl-3H-quin-azolin-4-one; 3-(2,6-dichlorophenyl)-5-methyl-2-(9H-purin-6-ylsulfanylmethyl)-3H-quinazolin-4-one; 3-(2-chlorophenyl)-6-fluoro-2-(9h-purin-6-ylsulfanylmethyl)-3H-quin-azolin-4-one; 5-chloro-3-(2-chlorophenyl)-2-(9H-purin-6-ylsulfanylmethyl)-3H-quin-azolin-4-one; 3-(2-chlorophenyl)-5-methyl-2-(9H-purin-6-ylsulfanylmethyl)-3H-quin-azolin-4-one; 3-(3-methoxyphenyl-2-(9H-purin-6-ylsulfanylmethyl-3H-quinazolin-4-o-ne; 3-(2-chlorophenyl)-5-fluoro-2-(9H-purin-6-ylsulfanylmethyl)-3H-quin-azolin-4-one; 3-benzyl-2-(9H-purin-6-ylsulfanylmethyl)-3H-quinazolin-4-one; 3-butyl-2-(9H-purin-6-ylsulfanylmethyl)-3H-quinazolin-4-one; 3-(2-chlorophenyl)-7-fluoro-2-(9H-purin-6-ylsulfanylmethyl)-3H-quin-azolin-4-one; 3-morpholin-4-yl-2-(9H-purin-6-ylsulfanylmethyl)-3H-quinazolin-4-on-e, acetate salt; 8-chloro-3-(2-chlorophenyl)-2-(9H-purin-6-ylsulfanylmethyl)-3H-quin-azolin-4-one; 3-(2-chlorophenyl)-6,7-difluoro-2-(9H-purin-6-ylsulfanylmethyl)-3H-quinazolin-4-one; 3-(2-methoxyphenyl-2-(9H-purin-6-ylsulfanylmethyl)-3H-quinazolin-4-one; 6-chloro-3-(2-chlorophenyl)-2-(9H-purin-6-ylsulfanylmethyl)-3H-quin-azolin-4-one; 3-(3-chlorophenyl)-2-(9H-purin-6-ylsulfanylmethyl)-3H-quinazolin-4-one; 2-(9H-purin-6-ylsulfanylmethyl)-3-pyridin-4-yl-3H-quinazolin-4-one; 3-(2-chlorophenyl)-2-(9H-purin-6-ylsulfanylmethyl)trifluoromethyl-3-H-quinazolin-4-one; 3-benzyl-5-fluoro-2-(9H-purin-6-ylsulfanylmethyl)-3H-quinazolin-4-o-ne; 3-(4-methylpiperazin-1-yl)-2-(9H-purin-6-ylsulfanylmethyl)-3H-quina-zolin-4-one, acetate salt; 3-(2-chlorophenyl)-6-hydroxy-2-(9H-purin-6-ylsulfanylmethyl)-3H-qui-nazolin-4-one; 5-fluoro-4-oxo-2-(9H-purin-6-ylsulfanylmethyl)-4H-quinazolin-3-yl]-acetic acid ethyl ester; 3-(2,4-dimethoxyphenyl)-2-(9H-purin-6-ylsulfanylmethyl)-3H-quinazol-in-4-one; 3-biphenyl-2-yl-5-chloro-2-(9H-purin-6-ylsulfanylmethyl)-3H-quinazo-lin-4-one; 2-(6-aminopurin-9-ylmethyl)-3-(2-isopropylphenyl)-5-methyl-3H-quina-zolin-4-one; 2-(6-aminopurin-9-ylmethyl)-5-methyl-3-o-tolyl-3H-quinazolin-4-one; 2-(6-aminopurin-9-ylmethyl)-3-biphenyl-2-yl-5-chloro-3H-quinazolin-4-one; 5-chloro-3-(2-methoxyphenyl)-2-(9H-purin-6-ylsulfanylmethyl)-3H-qui-nazolin-4-one; 2-(6-aminopurin-9-ylmethyl)-3-(2-fluorophenyl)-5-methyl-3H-quinazol-in-4-one; 2-(6-aminopurin-9-ylmethyl)-5-chloro-3-(2-fluorophenyl)-3H-quinazol-in-4-one; 2-(6-aminopurin-9-ylmethyl)-8-chloro-3-(2-chlorophenyl)-3H-quinazol-in-4-one; 2-(6-aminopurin-9-ylmethyl)-5-chloro-3-(2-chlorophenyl)-3H-quinazol-in-4-one; 2-(6-aminopurin-9-ylmethyl)-3-(2-chlorophenyl)-5-methyl-3H-quinazol-in-4-one; 2-(6-aminopurin-9-ylmethyl)-3-(2-chlorophenyl)-5-fluoro-3H-quinazol-in-4-one; 2-(6-aminopurin-9-ylmethyl)-3-benzyl-5-fluoro-3H-quinazolin-4-one; 2-(6-aminopurin-9-ylmethyl)-3-butyl-3H-quinazolin-4-one; 2-(6-aminopurin-9-ylmethyl)-3-morpholin-4-yl-3H-quinazolin-4-one; 2-(6-aminopurin-9-ylmethyl)-3-(2-chlorophenyl)-7-fluoro-3H-quinazol-in-4-one; 3-(2-chlorophenyl)-2-(9H-purin-6-ylsulfanylmethyl)-3H-quinazolin-4-one; 3-phenyl-2-(9H-purin-6-ylsulfanylmethyl)-3H-quinazolin-4-one; 2-(6-aminopurin-9-ylmethyl)-5-chloro-3-(2-isopropylphenyl)-3H-quina-zolin-4-one; and 2-(6-aminopurin-9-ylmethyl)-5-chloro-3-o-tolyl-3H-quinazolin-4-one. Additional inhibitors of PI3K delta that can be used in the practice of the present invention may be identified as disclosed in U.S. Pat. Nos. 5,858,753; 5,882,910; and 5,985,589, incorporated by reference in their entireties herein.

In the methods of the invention wherein a PI3Kδ subunit inhibitor is employed, it is preferred that the compound be at least about 10-fold selective, more preferably at least about 20-fold selective, even more preferably, at least about 50-fold selective for inhibition of PI3Kδ relative to other PI3K subunits in a cell-based assay.

In certain embodiments, the PI3K inhibitor is administered to an individual suffering from a condition associated with unwanted overproduction of type I IFN, such as autoimmune disease. An individual having an autoimmune disease is an individual with a recognizable symptom of an existing autoimmune disease or inflammatory disease.

Autoimmune diseases can be divided in two broad categories: organ-specific and systemic. Autoimmune diseases include, without limitation, rheumatoid arthritis (RA), systemic lupus erythematosus (SLE), psoriasis, type I diabetes mellitus, type II diabetes mellitus, multiple sclerosis (MS), immune-mediated infertility such as premature ovarian failure, scleroderrna, Sjogren's disease, vitiligo, alopecia (baldness), polyglandular failure, Grave's disease, hypothyroidism, polymyositis, pemphigus vulgaris, pemphigus foliaceus, inflammatory bowel disease including Crohn's disease and ulcerative colitis, autoimmune hepatitis including that associated with hepatitis B virus (HBV) and hepatitis C virus (HCV), hypopituitarism, graft-versus-host disease (GvHD), myocarditis, Addison's disease, autoimmune skin diseases, uveitis, pernicious anemia, and hypoparathyroidism.

Autoimmune diseases may also include, without limitation, Hashimoto's thyroiditis, Type I and Type II autoimmune polyglandular syndromes, paraneoplastic pemphigus, bullus pemphigoid, dermatitis-herpetiformis, linear IgA disease, epidermolysis bullosa acquisita, erythema nodosa, pemphigoid gestationis, cicatricial pemphigoid, mixed essential cryoglobulinemia, chronic bullous disease of childhood, hemolytic anemia, thrombocytopenic purpura, Goodpasture's syndrome, autoimmune neutropenia, myasthenia gravis, Eaton-Lambert myasthenic syndrome, stiff-man syndrome, acute disseminated encephalomyelitis, Guillain-Barre syndrome, chronic inflammatory demyelinating polyradiculoneuropathy, multifocal motor neuropathy with conduction block, chronic neuropathy with monoclonal gammopathy, opsonoclonus-myoclonus syndrome, cerebellar degeneration, encephalomyelitis, retinopathy, primary biliary sclerosis, sclerosing cholangitis, gluten-sensitive enteropathy, ankylosing spondylitis, reactive arthritides, polymyositis/dermatomyositis, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiutis obliterans, sarcoidosis, glomerulonephritis, and cryopathies. These conditions are well known in the medical arts and are described, for example, in Harrison's Principles of Internal Medicine, 14th ed., Fauci A S et al., eds., New York: McGraw-Hill, 1998.

The systemic disease SLE is characterized by the presence of antibodies to antigens that are abundant in nearly every cell, such as anti-chromatin antibodies, anti-splicesosome antibodies, anti-ribosome antibodies and anti-DNA antibodies. Consequently, the effects of SLE are seen in a variety of tissues, such as the skin and kidneys. Autoreactive T cells also play a role in SLE. For example, studies in a murine lupus model have shown that non-DNA nucleosomal antigens, e.g. histones, stimulate autoreactive T cells that can drive anti-DNA producing B cells. Increased serum levels of IFN-α has been observed in SLE patients and shown to correlate with both disease activity and severity, including fever and skin rashes, as well as essential markers associated with the disease process (e.g., anti-dsDNA antibody titers). It has also been shown that immune complexes present in the circulation could trigger IFN-α in these patients and, thus, maintain this chronic presence of elevated IFN-α. Two different types of immune complexes have been described to trigger IFN-α from human PDC: DNA/anti-DNA antibody complexes and RNA/anti-ribonucleoprotein-RNA antibody complexes. Because DNA is a ligand of TLR-9 and RNA a ligand for TLR-7/8, it is expected that these two pathways utilize TLR-9 and TLR-7/8 signalling, respectively, in order to chronically induce IFN-α and thus participate in the etiopathogenesis of SLE. Accordingly, specifically inhibitors of the PI3K δ subunit which are effective in inhibiting TLR-7/8 and TLR-9 responses may be particularly effective in treating SLE.

In certain embodiments, an individual is at risk of developing an autoimmune disease and a PI3K inhibitor (e.g., delta subunit specific PI3K inhibitor) is administered in an amount effective to delay or prevent the autoimmune disease. Individuals at risk of developing an autoimmune disease includes, for example, those with a genetic or other predisposition toward developing an autoimmune disease. In humans, susceptibility to particular autoimmune diseases is associated with HLA type with some being linked most strongly with particular MHC class II alleles and others with particular MHC class I alleles. For example, ankylosing spondylitis, acute anterior uveitis, and juvenile rheumatoid arthritis are associated with HLA-B27, Goodpasture's syndrome and MS are associated with HLA-DR2, Grave's disease, myasthenia gravis and SLE are associated with HLA-DR3, rheumatoid arthritis and pemphigus vulgaris are associated with HLA-DR4 and Hashimoto's thyroiditis is associated with HLA-DR5. Other genetic predispositions to autoimmune diseases are known in the art and an individual can be examined for existence of such predispositions by assays and methods well known in the art. Accordingly, in some instances, an individual at risk of developing an autoimmune can be identified.

As described herein, since PI3K inhibitors particularly inhibit production of type I IFN, methods of suppressing an unwanted immune response to an immunostimulatory nucleic acid in an individual are also provided.

Animal models for the study of autoimmune disease are known in the art. For example, animal models which appear most similar to human autoimmune disease include animal strains which spontaneously develop a high incidence of the particular disease. Examples of such models include, but are not limited to, the nonobese diabetic (NOD) mouse, which develops a disease similar to type 1 diabetes, and lupus-like disease prone animals, such as New Zealand hybrid, MRL-Fas^{1pr} and BXSB mice. Animal models in which an autoimmune disease has been induced include, but are not limited to, experimental autoimmune encephalomyelitis (EAE), which is a model for multiple sclerosis, collagen-induced arthritis (CIA), which is a model for rheumatoid arthritis, and experimental autoimmune uveitis (EAU), which is a model for uveitis. Animal models for autoimmune disease have also been created by genetic manipulation and include, for example, IL-2/IL-10 knockout mice for inflammatory bowel disease, Fas or Fas ligand knockout for SLE, and IL-1 receptor antagonist knockout for rheumatoid arthritis.

Accordingly, animal models standard in the art are available for the screening and/or assessment for activity and/or effectiveness of the methods and compositions of the invention for the treatment of autoimmune disorders.

In certain embodiments, the individual suffers from a disorder associated with a chronic inflammatory response. Administration of a PI3K delta subunit inhibitor results in immunomodulation, decreasing levels of Type I IFN, which may result in a reduction of the inflammatory response. Immunoregulation of individuals with the unwanted immune response associated the described disorders results in a reduction or improvement in one or more of the symptoms of the disorder.

Other embodiments of the invention relate to immunoregulatory therapy of individuals having been exposed to or infected with a virus. Administration of a PI3K delta subunit inhibitor to an individual having been exposed to or infected with a virus results in suppression of virus induced Type I IFN production. Cytokines produced in response to a virus can contribute to increased proinflammatory situation that can be deleterious for the host. Suppression of virus-induced Type I IFN production may serve to limit or prevent overwhelming inflammatory response.

The methods of the invention may be practiced in combination with other therapies which make up the standard of care for the disorder, such as administration of anti-inflammatory agents such as systemic corticosteroid therapy (e.g., cortisone).

In some situations, peripheral tolerance to an autoantigen is lost (or broken) and an autoimmune response ensues. For example, in an animal model for EAE, activation of antigen presenting cells (APCs) through the innate immune receptor TLR9 or TLR4 was shown to break self-tolerance and result in the induction of EAE (Waldner et al. (2004) J. Clin. Invest. 113:990-997).

Accordingly, in some embodiments, the invention provides methods for suppressing or reducing TLR (e.g., TLR7, TLR8 and/or TLR9) dependent cell stimulation. Administration of a PI3K delta subunit inhibitor results in decreased levels of Type I IFN.

### Administration

The PI3K delta subunit inhibitor can be administered in combination with other pharmaceutical agents, as described herein, and can be combined with a physiologically acceptable carrier thereof.

As with all compositions for modulation of an immune response, the effective amounts and method of administration of the particular PI3K delta subunit inhibitor formulation can vary based on the individual, what condition is to be treated and other factors evident to one skilled in the art. Factors to be considered include whether or not the PI3K inhibitor will be administered with or covalently attached to a delivery molecule, route of administration and the number of doses to be administered. Such factors are known in the art and it is well within the skill of those in the art to make such determinations without undue experimentation. A suitable dosage range is one that provides the desired suppression of IFN-α, for example in autoimmune disorders or in response to an immunostimulatory nucleic acid. Generally, dosage is determined by the amount of PI3K delta subunit inhibitor administered to the patient. Useful dosage ranges may be, for example, from about any of the following: 0.001 to 25 μM, 0.05 to 10 μM, 0.08 to 0.15 μM, 0.15 to 0.62 μM, 0.62 to 1.25 μM, 1 μM to 2 μM, 2 to 4 μM, and 1.25 to 5 μM. The absolute amount given to each patient depends on pharmacological properties such as bioavailability, clearance rate and route of administration.

The effective amount and method of administration of the particular PI3K delta subunit inhibitor or formulation comprising the inhibitor can vary based on the individual patient, desired result and/or type of disorder, the stage of the disease and other factors evident to one skilled in the art. The route(s) of administration useful in a particular application are apparent to one of skill in the art. Routes of administration include but are not limited to topical, dermal, transdermal, transmucosal, epidermal, parenteral, gastrointestinal, and naso-pharyngeal and pulmonary, including transbronchial and transalveolar. The absolute amount given to each patient depends on pharmacological properties such as bioavailability, clearance rate and route of administration.

As described herein, tissues in which unwanted Type I IFN production is occurring or is likely to occur are preferred targets for the PI3K delta subunit inhibitor. Thus, administration of PI3K delta subunit inhibitor to lymph nodes, spleen, bone marrow, blood, as well as tissue exposed to virus, are preferred sites of administration.

The present invention provides PI3K delta subunit inhibitor and PI3K delta subunit inhibitor formulations suitable for topical application including, but not limited to, physiologically acceptable implants, ointments, creams, rinses and gels. Exemplary routes of dermal administration are those which are least invasive such as transdermal transmission, epidermal administration and subcutaneous injection.

Transdermal administration is accomplished by application of a cream, rinse, gel, etc. capable of allowing the PI3K delta subunit inhibitor to penetrate the skin and enter the blood stream. Compositions suitable for transdermal administration include, but are not limited to, pharmaceutically acceptable suspensions, oils, creams and ointments applied directly to the skin or incorporated into a protective carrier such as a transdermal device (so-called “patch”). Examples of suitable creams, ointments etc. can be found, for instance, in the Physician's Desk Reference. Transdermal transmission may also be accomplished by iontophoresis, for example using commercially available patches which deliver their product continuously through unbroken skin for periods of several days or more. Use of this method allows for controlled transmission of pharmaceutical compositions in relatively great concentrations, permits infusion of combination drugs and allows for contemporaneous use of an absorption promoter.

Parenteral routes of administration include but are not limited to electrical (iontophoresis) or direct injection such as direct injection into a central venous line, intravenous, intramuscular, intraperitoneal, intradermal, or subcutaneous injection. Formulations of PI3K delta subunit inhibitor(s) suitable for parenteral administration are generally formulated in USP water or water for injection and may further comprise pH buffers, salts bulking agents, preservatives, and other pharmaceutically acceptable excipients. Immunoregulatory polynucleotide for parenteral injection may be formulated in pharmaceutically acceptable sterile isotonic solutions such as saline and phosphate buffered saline for injection.

Gastrointestinal routes of administration include, but are not limited to, ingestion and rectal routes and can include the use of, for example, pharmaceutically acceptable powders, pills or liquids for ingestion and suppositories for rectal administration.

Naso-pharyngeal and pulmonary administration include are accomplished by inhalation, and include delivery routes such as intranasal, transbronchial and transalveolar routes. The invention includes formulations of PI3K delta subunit inhibitor(s) suitable for administration by inhalation including, but not limited to, liquid suspensions for forming aerosols as well as powder forms for dry powder inhalation delivery systems. Devices suitable for administration by inhalation of PI3K delta subunit inhibitor formulations include, but are not limited to, atomizers, vaporizers, nebulizers, and dry powder inhalation delivery devices.

As is well known in the art, solutions or suspensions used for the routes of administration described herein can include any one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates' or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

As is well known in the art, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

As is well known in the art, sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The above-mentioned compositions and methods of administration are meant to describe but not limit the methods of administering the formulations of PI3K delta subunit inhibitor of the invention. The methods of producing the various compositions and devices are within the ability of one skilled in the art and are not described in detail here.

Analysis (both qualitative and quantitative) of the activity of a PI3K delta subunit inhibitor in suppression of Type I IFN production can be by any method described herein or known in the art. Measurement of numbers of specific types of cells can be achieved, for example, with fluorescence-activated cell sorting (FACS). Measurement of maturation of particular populations of cells can be achieved by determining expression of markers, for example, cell surface markers, specific for particular stage of cell maturation. Cell marker expression can be measured, for example, by measuring RNA expression or measuring cell surface expression of the particular marker by, for example, FACS analysis. Measuring maturation of dendritic cells can be performed for instance as described in Hartmann et al. (1999) Proc. Natl. Acad. Sci. USA 96:9305-9310. Cytokine concentrations can be measured, for example, by ELISA. These and other assays to evaluate suppression of an immune response, including an innate immune response, are well known in the art.

### Kits of the Invention

The invention also provides kits. In certain embodiments, the kits of the invention generally comprise one or more containers comprising a PI3K delta subunit inhibitor. The kits may further comprise a suitable set of instructions, generally written instructions, relating to the use of the PI3K delta subunit inhibitor for any of the methods described herein (e.g., suppression of Type I IFN production, ameliorating one or more symptoms of an autoimmune disease, ameliorating a symptom of chronic inflammatory disease, decreasing Type I IFN in response to a virus).

The kits may comprise PI3K delta subunit inhibitor packaged in any convenient, appropriate packaging. For example, if the PI3K delta subunit inhibitor is a dry formulation (e.g., freeze dried or a dry powder), a vial with a resilient stopper is normally used, so that the PI3K delta subunit inhibitor may be easily resuspended by injecting fluid through the resilient stopper. Ampoules with non-resilient, removable closures (e.g., sealed glass) or resilient stoppers are most conveniently used for liquid formulations of PI3K delta subunit inhibitor. Also contemplated are packages for use in combination with a specific device, such as an inhaler, nasal administration device (e.g., an atomizer), a syringe or an infusion device such as a minipump.

The instructions relating to the use PI3K delta subunit inhibitor generally include information as to dosage, dosing schedule, and route of administration for the intended method of use. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The following Examples are provided to illustrate, but not limit, the invention.

### EXAMPLES

### Example 1

### Activation of the PI3K Pathway in Human PDC

To assess the activity of PI3K in primary human pDC, we measured the phosphorylation of Akt/PKB (P-Akt), a downstream target of PI3K (11).

Buffy coats were obtained either from the Stanford Blood Center (Palo Alto, Calif.) and cells were used under internal Institutional Review Board-approved protocols or from adult healthy donors (Saint-Antoine Crozatier Blood Bank, Paris, France) where all donors signed informed consent to allow the use of their blood for research purposes. This study was approved by the Institut Curie Internal Review Board and by the French National Blood Agency (Etablissement Français du Sang). PDC were isolated either by using positive selection using BDCA-4 conjugated beads or by using negative depletion (Miltenyi Biotech) as previously described (35). PDC were 94-99% BDCA2⁺ CD123⁺ as determined by flow cytometry.

Oligonucleotides CpG-C C274 and CpG-A D19 were prepared as previously described (3). UV inactivated HSV-1 was a kind gift from R. Pyles, University of Texas Medical Branch, Galveston, Tex. Heat inactivated Influenza virus (H1N1, strain A/PR/8/34) was obtained from ATCC (Manassas, Va.).

Purified PDC were cultured with 1 μM CpG-C or 2 MOI Flu for 20 min or 90 min with or without the PI3K inhibitor (LY) at 1 μM. Cells were immediately fixed with 4% of paraformaldehyde for 15 min a 37 C. Cells were then washed, permeabilized with PermBuffer III (BD bioscience) for 30 minutes on ice and stained with Alexa-647 anti-human AKT (pS473) (BD Bioscience) for 30 min and then analyzed by flow cytometry.

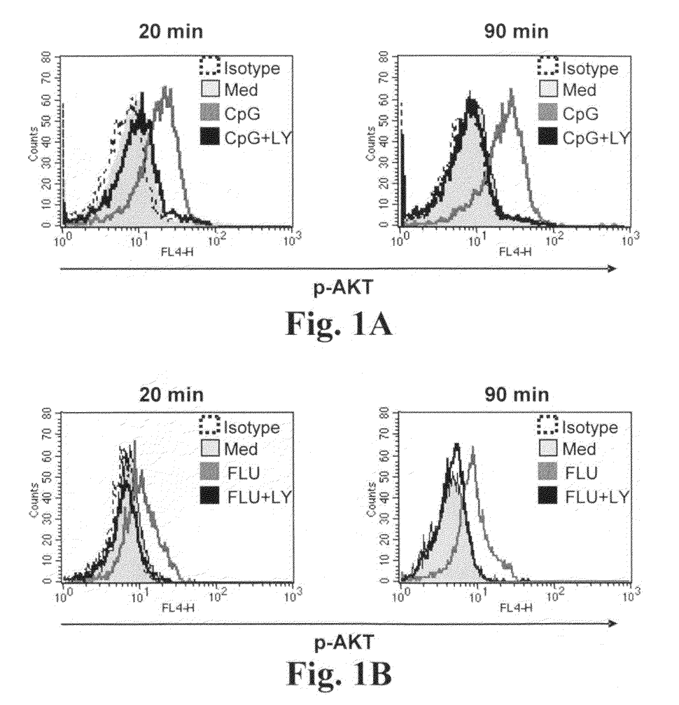

As shown in FIG. 1, P-Akt was not detected at significant levels in freshly sorted pDC, and was not induced by serum-containing medium, as opposed to other cell culture systems where serum could induce PI3K activation (18). However, P-Akt was upregulated after both 20 and 90 minutes of culture in the presence of CpG-C or Flu (FIGS. 1A and B). This increase was PI3K-dependent, since it could be blocked by the specific PI3K inhibitor LY294002 (LY) at both time points and for both TLR ligands (FIGS. 1A and B). Thus, TLR-ligands induce PI3K-dependent Akt phosphorylation in primary human pDC.

Our data is consistent with results indicating that TLR-9 signaling leads to PI3K activation in different cell types, such as CD4+ T cells (16), murine macrophages (17) or splenic DC (19). Following TLR-9 triggering, Akt phosphorylation was observed 30 minutes after CpG stimulation (16, 19), comparable to our data on human pDC. This rapid response, together with the ability of MyD88 to associate to the p85 subunit of PI3K (16), support a direct TLR-induced activation of PI3K rather than indirect activation through a TLR-induced autocrine loop.

### Example 2

### Selective Involvement of PI3-Kinase for Type I IFN Production by TLR-Activated pDC

### A. PI3K Inhibition Selectively Inhibits TLR7 and TLR9 Mediated IFN-α Response in Human PDC

The selective inhibition of PI3K in TLR2, 4 and 9 stimulated mouse DC and macrophages enhanced IL-12 production suggests that PI3K may negatively regulate TLR-induced inflammatory response in APC (13). To address the role of PI3K in human pDC, purified cells were stimulated with TLR9 (CpG-C, HSV) or TLR7 (Flu) ligands, with or without the pharmacological inhibitors of PI3K, LY and wortmannin.

PI3K inhibitors LY294002 (LY) and wortmannin and NF-kB inhibitor (IKK-2 IV) were purchased from Calbiochem. PI3Kγ inhibitor (AS 604850) was purchased from Echelon. PI3Kδ inhibitor (IC 87114) was synthesized as previously described (Patent application US 2005/0261317 A1).

Purified human pDC as described in Example 1 were cultured with CpG-C (1 μM), HSV (5 MOI) or Flu (1 MOI) viruses either alone or in combination with various concentration of PI3K inhibitor LY (μM) for 16 hr and IFN-α production was evaluated by ELISA. For ELISA, human IFN-α, IL-6 and TNF-α ELISA set were purchased from PBL Biomedical Laboratories (Piscataway, N.J.) and anti-CD123, anti-CD80, anti-CD86 anti-CD71 and anti-CD 107a from BD Bioscience, anti-BDCA-2 from Miltenyi Biotech (Auburn, Calif.).

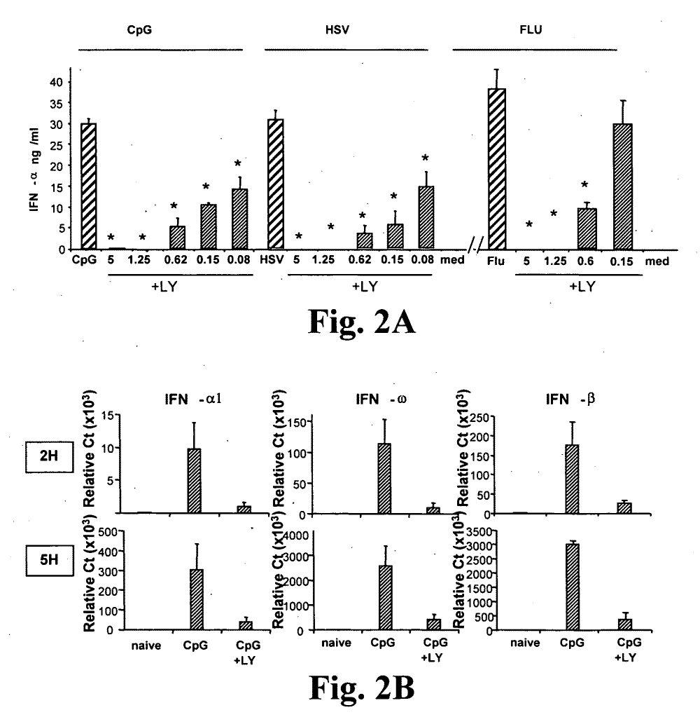

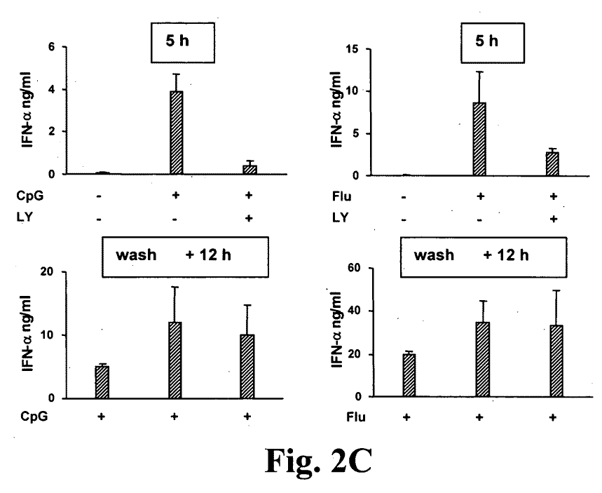

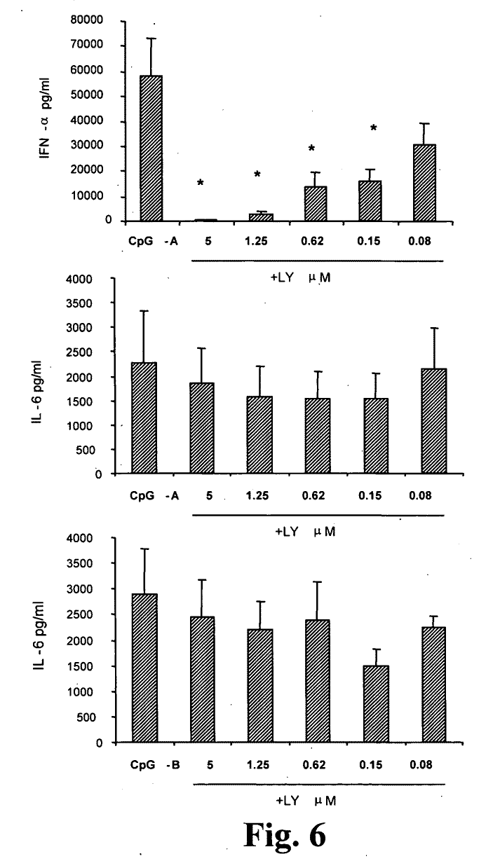

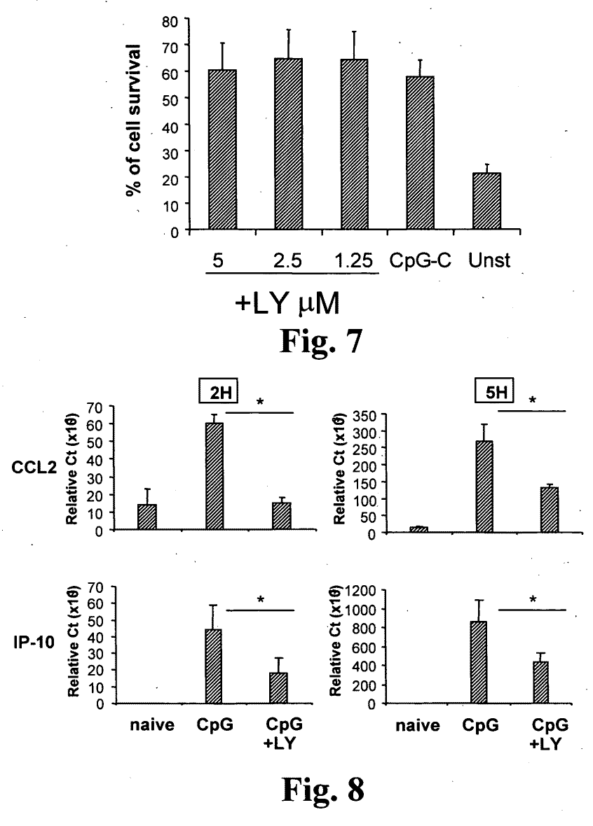

The TLR ligands induced high levels (20-30 ng/ml) of IFN-α production by freshly sorted pDC (FIG. 2A). This response was strongly inhibited by LY in a dose-dependent manner with a maximal effect at 1.25 μM of LY for both TLR7 and TLR9 ligands (FIG. 2A). A 50% inhibition of IFN-α was still observed with LY concentrations as low as 0.08 μM for TLR9 (FIG. 2A). Similarly, strong inhibition of IFN-α was observed in CpG-A stimulated pDC (FIG. 6). Importantly, no negative effect on pDC viability was observed at any of the concentrations used (FIG. 7). Similar results were obtained with wortmannin, a general PI3K inhibitor.

Because specificity of signaling inhibitors can be an issue, especially in cultures exceeding several hours, we also performed two types of experiments to exclude non-specific effects due to the potential toxicity of using PI3K inhibitors that could affect important functions of pDC. First, we cultured pDC for shorter periods of 2 and 5 hours, and analyzed the ability of PI3K inhibitors to inhibit the IFN-α response at the transcriptional level. In particular, purified PDC were cultured with CpG-C (1 μM) alone or in the presence of LY inhibitor (2 μM) for 2 hours and 5 hours. The expression levels of IFN-α, IFN-ω and IFN-β were measured by real-time quantitative PCR (TaqMan) analysis. PCR reactions were performed as described previously (3). In brief, threshold cycle (CT) values for each gene were normalized to the housekeeping gene Ubiquitin or β-actin using the formula Eq. 1.8^{(HSKGENE)} (100,000), where HSK is the mean CT of triplicate housekeeping gene runs, GENE is the mean CT of duplicate runs of the gene of interest, and 100,000 is arbitrarily chosen as a factor to bring all values above 0.

After 2 hours, we detected significant IFN-α, IFN-β and IFN-ω mRNA in the presence of CpG-C, which was almost completely inhibited by LY (FIG. 2B). The same magnitude of inhibition was observed at 5 hours of culture (FIG. 2B).

Second, we attempted to reverse the inhibition of IFN-α production by washing out the inhibitor. In particular, purified pDC were cultured with CpG-C or Flu, either alone or in the presence of LY inhibitor. The supernatants were collected after 5 hours after which the cells were washed twice and restimulated with CpG-C or Flu for another 12 hours. IFN-α production was evaluated by ELISA as described above.

After 5 hours of culture, CpG-induced IFN-α production was inhibited in the presence of LY (FIG. 2C). Washing out the inhibitor after the first 5 hours enabled pDC to recover their ability to produce large amounts of IFN-α during the subsequent 12 hours (FIG. 2C).

Autocrine IFN-α signaling has been shown to account for a portion of the induction of chemokines, such as CCL2 and IP-10, in response to TLR-9 activation (20). Consistent with a strong inhibition of IFN-α production, PI3K inhibition induced a 70% reduction in the expression of CCL2 and IP-10 in CpG-activated pDC (FIG. 8).

B. P13K Inhibition does not Affect Inflammatory Cytokines or Maturation of PDC in Response to TLR7/9 Triggering

In addition to large amounts of type I IFNs, TLR activation of pDC can induce the production of pro-inflammatory cytokines such as IL-6 and TNF-α. To assess IL-6 and TNF-α production, purified PDC were cultured with CpG-C ISS (1 μM), HSV (5 MOI) or Flu (1 MOI) viruses either alone or in combination with various concentration of PI3K inhibitor LY for 2, 5 or 16 hours. IL-6 and TNF-α production was evaluated by ELISA; by real-time PCR; and cells were characterized for CD80 and CD86 expression by flow cytometry analysis.

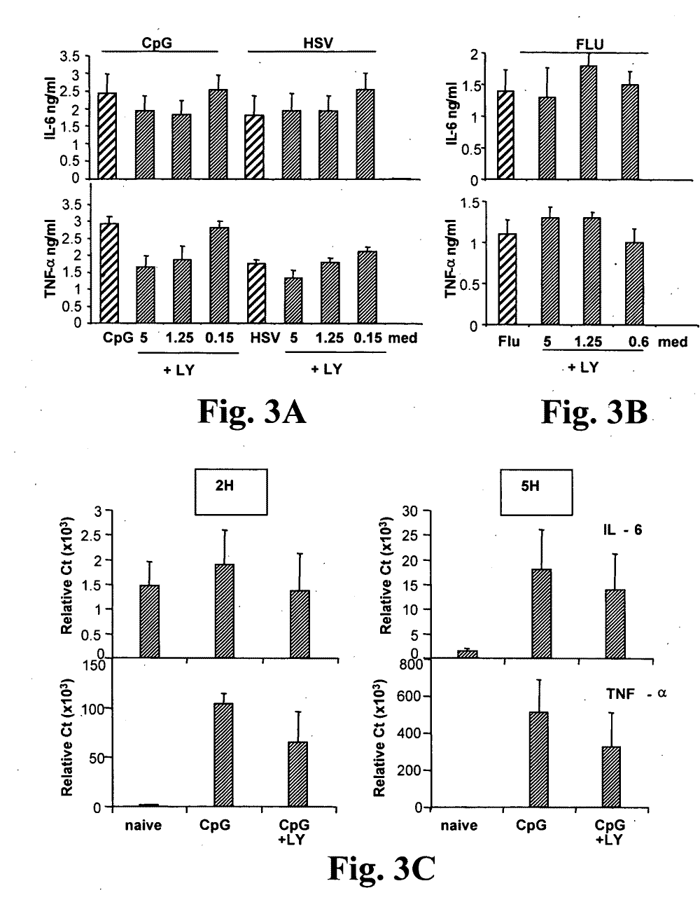

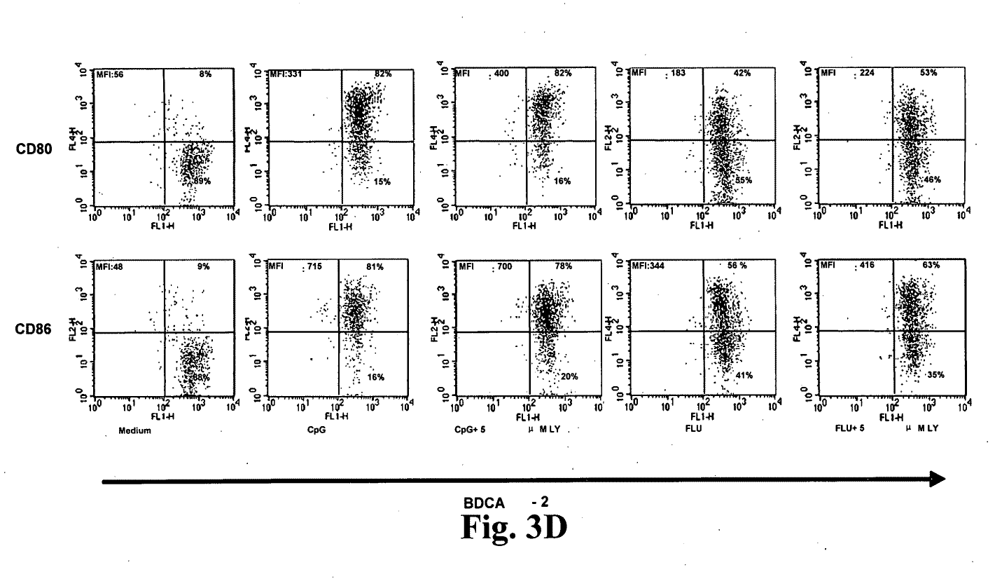

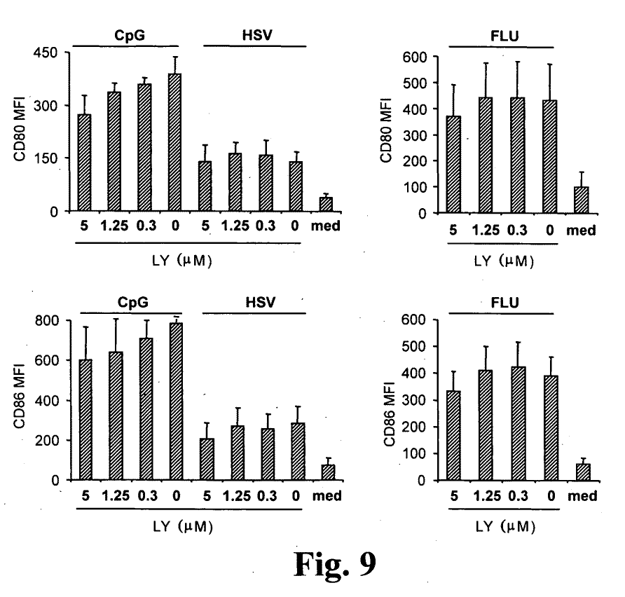

By contrast to the strong inhibition of type I IFN, TNF-α and IL-6 production by pDC in response to both TLR9 or TLR7 ligands was not significantly affected by the addition of LY, even at high (5 μM) concentrations of the inhibitor (FIGS. 3A, 3B and 6). This was confirmed at the transcriptional level (FIG. 3C). Similarly, the pDC differentiation into mature DC, as assessed by surface expression of costimulatory molecules CD80 and CD86, was not significantly affected by PI3K inhibitors (FIGS. 3D and 9).

These data demonstrate that PI3Kinase is selectively involved in the IFN-α pathway but not in the signaling events required for TNF-α or maturation induction. Moreover, they show that important functional pathways are conserved in pDC despite PI3K inhibition, which, together with the conserved viability of pDC, demonstrate that the observed effect on IFN-α was not due to overall toxicity of the inhibitor.

### Example 3

### Identification of the PI3K Subunit Involved in Production of Type I IFN

To further define the function of the different subunits of PI3K in human pDC, we addressed (i) their expression profile and (ii) their respective contribution to regulate type I IFN in pDC.

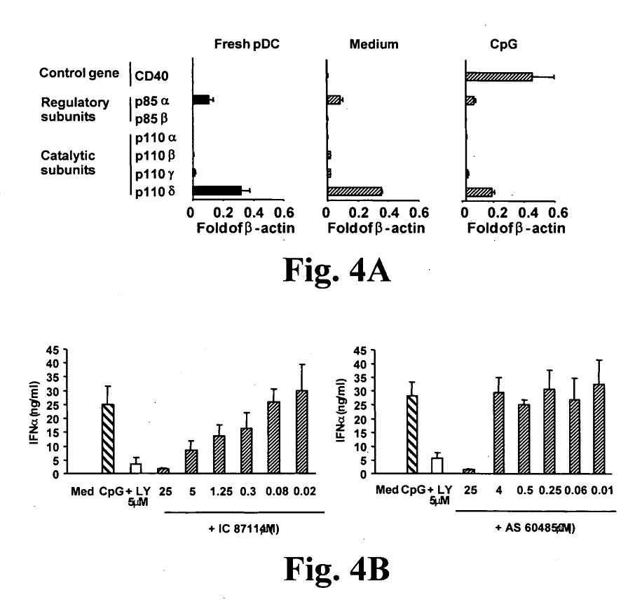

First, we showed that freshly purified and activated pDC preferentially expressed the p85α regulatory subunit and the p110δ catalytic subunit (FIG. 4A). Second, we showed that the PI3K 6-specific inhibitor IC87114 (25) inhibited IFN-α production in a dose-dependent manner (FIG. 4B). By contrast, when cultured pDC in the presence of the PI3K γ-specific inhibitor AS604850, we did not observe any effect on IFN-α production unless used at high concentration (>20 μM) where its specificity for the gamma subunit is lost (26).

These results demonstrate that the PI3K δ subunit is the essential subunit involved in the production of IFN-α by pDC.

### Example 4

### PI3kinase Inhibition does not Affect the Uptake and Endosomal Location of CpG ODN

CpG-ODN require both uptake and localization into appropriate endosomal compartments in order to signal through TLR9. PI3K is important for phagocytosis and endocytosis in various cellular models (27) partly by contributing to phagosome formation and maturation (27, 28). In addition, it was previously shown that blocking PI3K resulted in a complete blockade of CpG-ODN uptake in mouse myeloid DC and TLR9-transfected HEK 293 cells (29).

To test the role of PI3K in uptake in PDC, we tested the effect of PIK3K inhibitors on fluorescent CpG ODN, as measured by flow cytometry. Briefly, purified PDC were cultured for 3 hours either alone or with 0.5 μM CpG-C/Alexa-488 alone or in the presence of LY or wortmannin inhibitors (5 μM) and internalization of the fluorescent ISS was evaluated by flow cytometry. Surface bound fluorescence was quenched with a solution of 100 μg/ml trypan blue in PBS.

As shown in FIG. 1A, inhibiting PI3K with LY or wortmannin did not have any effect on CpG uptake.

Furthermore, we and others have shown recently that the nature of pDC response to TLR9 strongly depends on the intracellular compartment where the interaction receptor/ligand occurs (4, 5). In human pDC, the production of IFN-α is associated with trafficking of CpG in the early endosomal compartment while maturation in antigen presenting cells required accumulation of the CpG within the late endosomal compartment (5).

We therefore investigated whether PI3K inhibition would interfere with the localization of the CpG in the early endosome compartment, a situation that is predicted to hamper IFN-α response. Evaluation of intracellular localization of CpG was performed essentially as previously described (5). Purified PDC were cultured with fluorescent CpG-C alone or in the presence of LY inhibitor (5 μM) for 3 h. Cells were then fixed and stained intracellularly with anti-transferrin receptor (TfR) or anti-LAMP1 (LP1) antibodies and imaged by confocal microscopy.

Images were acquired using a ZEISS LSM 510 META confocal microscope and a 63×/1.4 N.A. objective, with the pinhole set for a section thickness of 0.8 μm. Images were acquired sequentially using separate laser excitation to avoid any cross-talk between the fluorophore signals.

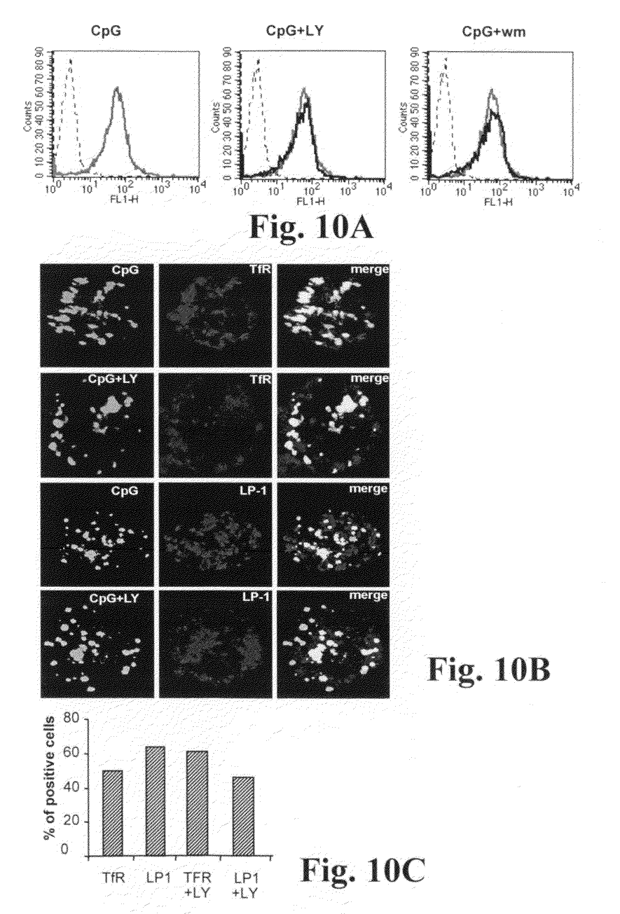

Transferrin receptor (TfR) and Lamp-I were used as markers of early and late endosomes, respectively. As previously described (5), in the absence of PI3K inhibition, fluorescent CpG-C co-localized with TfR- as well as Lamp-containing endosomal compartments (FIG. 10B). This pattern of distribution was not affected by PI3K inhibitors, indicating that PI3K does not interfere with the intra-cellular trafficking of CpG in primary pDC (FIGS. 10B and 10C).

Thus, PI3K inhibition did not prevent the localization of the CpG in the early endosome that is essential for triggering IFN-α at time points where inhibition of IFN-α was almost complete by gene expression analysis (FIG. 2B). Furthermore, the concentration of LY was similar to the one used to demonstrate inhibition of IFN-α at similar time-point of the stimulation (FIG. 2B).

These data show that PI3K does not interfere with the uptake and distribution of the TLR ligands and indicate that PI3K plays a role in the signaling pathway downstream of TLR7 or 9 activation.

### Example 5

### PI3kinase is Required for IRF-7 Nuclear Translocation but not NF-kB Phosphorylation in TLR-Activated pDC

In mouse pDC, IFN-α production depends on the activation and translocation of IRF-7 to the nucleus (6). Moreover the strong upregulation of IRF-7 messenger was suggested to be key for the high magnitude of IFN-α response upon TLR7/9 ligation in human pDC (30). We thus investigated whether PI3K alters this pathway by looking at both transcriptional upregulation of IRF-7 and its ability to migrate to the nucleus upon activation.

First, purified PDC (1×10⁵) were cultured with or without CpG-C ISS (1 μM) alone or in the presence of LY inhibitor (5 μM). IRF-7 expression was evaluated 2 hours and 5 hours after stimulation by real time PCR by measuring mRNA levels.

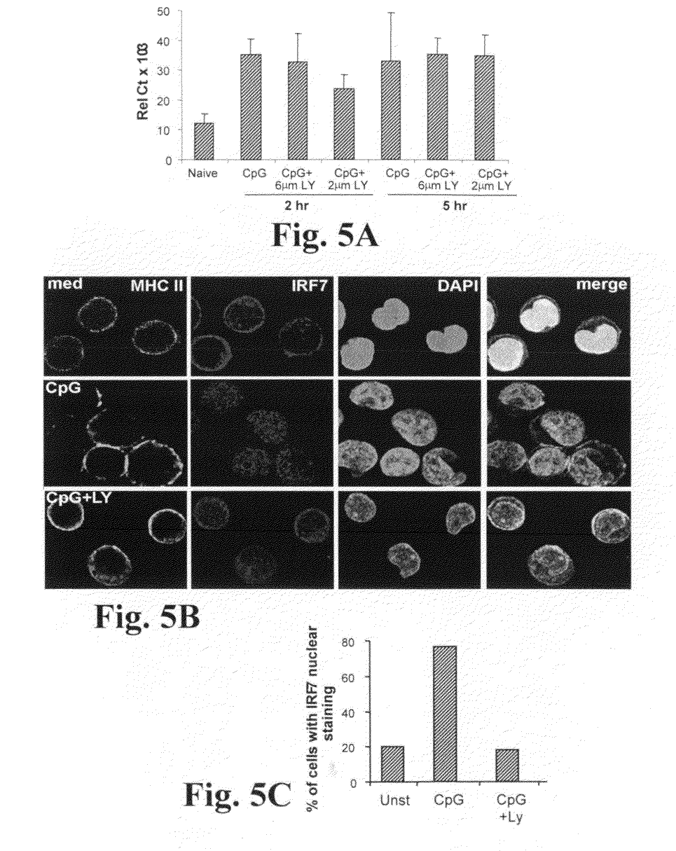

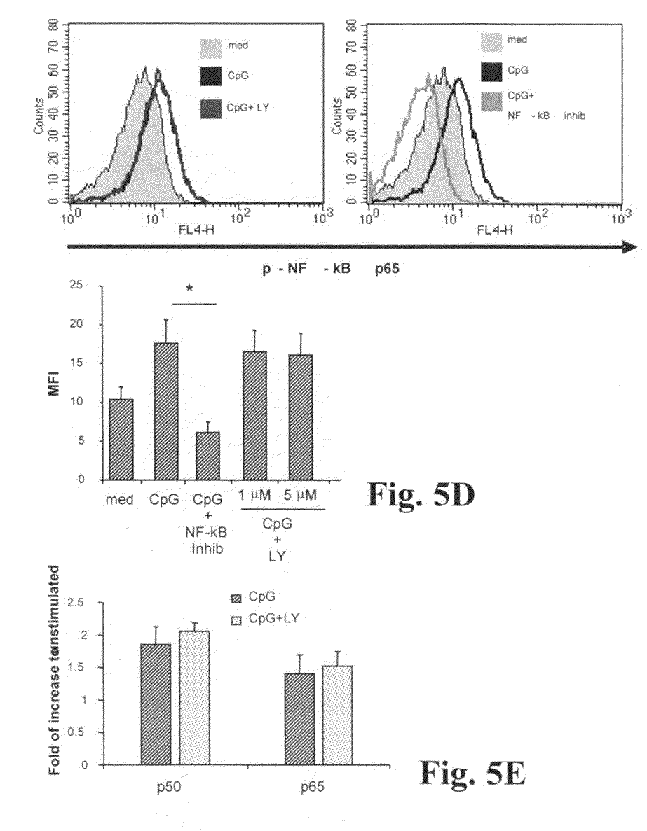

As shown in FIG. 5A, freshly sorted pDC constitutively expressed IRF-7 mRNA, and that its level was increased 2 and 5 hours after CpG stimulation. This transcriptional upregulation of IRF-7 was not affected in the presence of PI3K inhibitor (FIG. 5A).

We then studied the nuclear translocation of IRF-7. Briefly, 2×10⁵ purified PDC were left untreated or stimulated with CpG alone or in the presence of LY inhibitor for 3 hours. Cells were visualized using confocal microscopy using the membrane staining of Class II molecule (FITC) while the nucleus was identified using DAPI. IRF-7 nuclear translocation was visualized by immunofluorescence with IRF-7 antibody (Alexa 555/red). In particular, IRF-7 detection was performed as follows: purified PDC (2×10⁵/200 ul in 96 well round bottom) were stimulated with 1 μM of CpG-A or CpG-C either alone or in the presence of the PI3K inhibitor LY294002 (5 μM) for 3 hours. Cells were first stained with anti-human MHC Class II-FITC and subsequently fixed with 2% paraformaldehyde and then permeabilized with 100% ice cold methanol for 10 min at −20 C. Samples were then labeled with rabbit polyclonal anti-human IRF-7 (Santa Cruz Biotechnology). Anti rabbit IgG alexa 555 (Molecular Probes) was used as secondary antibody. Cells were seeded on glass slides by cytospin and mounted using Prolong antifade with DAPI (Molecular Probes).

As shown in FIG. 5B, the IRF-7 protein was expressed in the cytoplasm of unstimulated pDC and did not colocalize with the DAPI nuclear staining. In addition, MHC class II surface staining used to visualize the pDC, showed that, after stimulation with CpG, the majority of IRF-7 translocated to the nucleus, as assessed by the colocalization of the IRF-7 (2^{nd} panels from left of FIG. 5B) and DAPI (2^{nd} panels from right of FIG. 5B) stainings, as well as the reduction of detectable IRF-7 staining in the cytoplasmic compartment (FIG. 5B). This process was dramatically decreased in the presence of a PI3K inhibitor, with the majority of the IRF-7 staining remaining in the cytoplasm (FIG. 5B).

FIG. 5C shows analysis of between 50-70 cells from at least 4 different donors that were analyzed for IRF7 translocation in the nuclei. Cells were considered positive when at least 20% of the IRF-7 fluorescence was localized in the nucleus. The total number of cells showing nuclear staining of IRF-7 returned to baseline levels in the presence of LY (FIG. 5C). Similar results were obtained with both IFN inducing class of CpG, A and C, as well as with HSV virus.

Furthermore, as shown in FIGS. 2 and 3, PI3K appears to be essential for IFN-α response but not for other inflammatory cytokines and DC differentiation, two responses that were shown in the mouse to be mostly NF-kB-dependent (31). Accordingly, purified PDC were cultured with CpG-C ISS (1 μM) with or without PI3K inhibitor LY (1 μM or 5 μM) or NF-kB inhibitor (0.5 μM) for 90 min and analyzed by NF-kB flow cytometry. As described above, negatively purified pDC were stimulated with CpG-C and cells were immediately fixed with 4% of paraformaldehyde for 15 min a 37 C. Cells were then washed, permeabilized with PermBuffer III (BD bioscience) for 30 minutes on ice and stained with either Alexa-647 anti-human NF-kB p65 (pS529) for 30 min and then analyzed by flow cytometry.

We show that in parallel to IRF-7 activation, CpG-C also induced phosphorylation of NF-kB, as assessed by flow cytometry (FIG. 5D). Interestingly, although the NF-kB phosphorylation was inhibited using a specific NF-kB inhibitor, we did not observe any significant effect of the PI3K inhibitor LY (FIG. 5D).

To confirm that the NF-kB pathway was not affected following PI3K inhibition, we analyzed pDC nuclear extracts for the binding activity of NF-kB p50 and p65 subunits. Purified PDC were cultured with CpG-C ISS (1 μM) with or without PI3K inhibitor LY (5 μM) for 4 hours and nuclear extracts were analyzed or the binding activity of NF-kB p50 and p65 family members. Negatively purified pDC were stimulated and nuclear extracts were prepared. NF-κB activities were measured using TransAM NF-κB Kits (Active Motif) according to the manufacturer's instructions.

No difference was detected in the absence or presence of LY (FIG. 5E), indicating an absence of cross-talk between the PI3K and NF-kB pathways in human pDC.

Our results provide a molecular link between PI3K activity and the regulation of type I IFN production by pDC and identify PI3K as an essential component of the pathway leading to IFN production in pDC. In addition, our results show that PI3K is a key component of the signal transduction pathway that controls IRF-7 nuclear translocation and subsequent type I IFN production by human pDC and indicate that PI3K is essential for pDC to respond properly to viruses by favoring early production of type I IFN.

The results presented herein allow for the manipulation of cells involved in pathological conditions, such as autoimmune or infectious diseases. The role of IFN-α in the development of auto-immunity, and pDC, through their production of high levels of type I IFN, have been implicated in the pathophysiology of various auto-immune diseases, such as systemic lupus erythematosus, psoriasis or Sjögren's disease (9, 10). Accordingly, PI3K inhibitors, in particular those targeting specifically the delta subunit, offer a unique way to selectively block type I IFN production while preserving NF-kB-dependent responses, which could have important pro-inflammatory or regulatory effects through modulation of T cell responses. Furthermore, delta subunit-specific PI3K inhibitors also limit general toxicity associated with non-specific PIK3 inhibitors.

### REFERENCES CITED

1. Liu, Y. J. 2005. IPC: professional type 1 interferon-producing cells and plasmacytoid dendritic cell precursors. Annu. Rev. Immunol. 23:275-306.
2. Colonna, M., G. Trinchieri, and Y. J. Liu. 2004. Plasmacytoid dendritic cells in immunity. Nat. Immunol. 5:1219-1226.
3. Duramad, O., K. L. Fearon, J. H. Chan, H. Kanzler, J. D. Marshall, R. L. Coffman, and F. J. Barrat. 2003. IL-10 regulates plasmacytoid dendritic cell response to CpG-containing immunostimulatory sequences. Blood 102:4487-4492.
4. Honda, K., Y. Ohba, H. Yanai, H. Negishi, T. Mizutani, A. Takaoka, C. Taya, and T. Taniguchi. 2005. Spatiotemporal regulation of MyD88-IRF-7 signalling for robust type-I interferon induction. Nature 434:1035-1040.
5. Guiducci, C., G. Ott, J. H. Chan, E. Damon, C. Calacsan, T. Matray, K. D. Lee, R. L. Coffinan, and F. J. Barrat. 2006. Properties regulating the nature of the plasmacytoid dendritic cell response to Toll-like receptor 9 activation. J. Exp. Med. 203:1999-2008.
6. Honda, K., H. Yanai, H. Negishi, M. Asagiri, M. Sato, T. Mizutani, N. Shimada, Y. Ohba, A. Takaoka, N. Yoshida, and T. Taniguchi. 2005. IRF-7 is the master regulator of type-I interferon-dependent immune responses. Nature 434:772-777.
7. Uematsu, S., S. Sato, M. Yamamoto, T. Hirotani, H. Kato, F. Takeshita, M. Matsuda, C. Coban, K. J. Ishii, T. Kawai, O. Takeuchi, and S. Akira. 2005. Interleukin-1 receptor-associated kinase-1 plays an essential role for Toll-like receptor (TLR)7- and TLR9-mediated interferon-{alpha} induction. J. Exp. Med. 201:915-923.
8. Hoshino, K., T. Sugiyama, M. Matsumoto, T. Tanaka, M. Saito, H. Hemmi, O. Ohara, S. Akira, and T. Kaisho. 2006. IkappaB kinase-alpha is critical for interferon-alpha production induced by Toll-like receptors 7 and 9. Nature 440:949-953.
9. Kanzler, K., F. J. Barrat, E. M. Hessel, and R. L. Coffman. 2007. Therapeutic targeting of innate immunity with Toll-like receptor agonists and antagonists. Nat. Med. 13:552-559.
10. Colonna, M. 2006. Toll-like receptors and IFN-alpha: partners in autoimmunity. J. Clin. Invest. 116:2319-2322.
11. Deane, J. A., and D. A. Fruman. 2004. Phosphoinositide 3-kinase: diverse roles in immune cell activation. Annu. Rev. Immunol. 22:563-598.
12. Rommel, C., M. Camps, and H. Ji. 2007. PI3K delta and PI3K gamma: partners in crime in inflammation in rheumatoid arthritis and beyond? Nat. Rev. Immunol. 7:191-201.
13. Fukao, T., and S. Koyasu. 2003. PI3K and negative regulation of TLR signaling. Trends Immunol. 24:358-363.
14. Fukao, T., M. Tanabe, Y. Terauchi, T. Ota, S. Matsuda, T. Asano, T. Kadowaki, T. Takeuchi, and S. Koyasu. 2002. PI3K-mediated negative feedback regulation of IL-12 production in DCs. Nat. Immunol. 3:875-881.
15. Williams, D. L., C. Li, T. Ha, T. Ozment-Skelton, J. H. Kalbfleisch, J. Preiszner, L. Brooks, K. Breuel, and J. B. Schweitzer. 2004. Modulation of the phosphoinositide 3-kinase pathway alters innate resistance to polymicrobial sepsis. J. Immunol. 172:449-456.
16. Gelman, A. E., D. F. LaRosa, J. Zhang, P. T. Walsh, Y. Choi, J. O, Sunyer, and L. A. Turka. 2006. The adaptor molecule MyD88 activates PI-3 kinase signaling in CD4+ T cells and enables CpG oligodeoxynucleotide-mediated costimulation. Immunity 25:783-793.
17. Sester, D. P., K. Brion, A. Trieu, H. S. Goodridge, T. L. Roberts, J. Dunn, D. A. Hume, K. J. Stacey, and M. J. Sweet. 2006. CpG DNA activates survival in murine macrophages through TLR9 and the phosphatidylinositol 3-kinase-Akt pathway. J. Immunol. 177:4473-4480.
18. Tsuruta, F., N. Masuyama, and Y. Gotoh. 2002. The phosphatidylinositol 3-kinase (PI3K)-Akt pathway suppresses Bax translocation to mitochondria. J. Biol. Chem. 277:14040-14047.
19. Park, Y., S. W. Lee, and Y. C. Sung. 2002. Cutting Edge: CpG DNA inhibits dendritic cell apoptosis by up-regulating cellular inhibitor of apoptosis proteins through the phosphatidylinositide-3′-OH kinase pathway. J. Immunol. 168:5-8.
20. Megjugorac, N. J., H. A. Young, S. B. Amrute, S. L. Olshalsky, and P. Fitzgerald-Bocarsly. 2004. Virally stimulated plasmacytoid dendritic cells produce chemokines and induce migration of T and NK cells. J. Leukoc. Biol. 75:504-514.
21. Lee, H. K., J. M. Lund, B. Ramanathan, N. Mizushima, and A. Iwasaki. 2007. Autophagy-dependent viral recognition by plasmacytoid dendritic cells. Science 315:1398-1401.
22. Barrat, F. J., T. Meeker, J. Gregorio, J. H. Chan, S. Uematsu, S. Akira, B. Chang, O. Duramad, and R. L. Coffman. 2005. Nucleic acids of mammalian origin can act as endogenous ligands for Toll-like receptors and may promote systemic lupus erythematosus. J. Exp. Med. 202:1131-1139.
23. Cao, W., D. B. Rosen, T. Ito, L. Bover, M. Bao, G. Watanabe, Z. Yao, L. Zhang, L. L. Lanier, and Y. J. Liu. 2006. Plasmacytoid dendritic cell-specific receptor ILT7-Fc epsilonRI gamma inhibits Toll-like receptor-induced interferon production. J. Exp. Med. 203:1399-1405.
24. Dzionek, A., Y. Sohma, J. Nagafune, M. Cella, M. Colonna, F. Facchetti, G. Gunther, I. Johnston, A. Lanzavecchia, T. Nagasaka, T. Okada, W. Vermi, G. Winkels, T. Yamamoto, M. Zysk, Y. Yamaguchi, and J. Schmitz. 2001. BDCA-2, a novel plasmacytoid dendritic cell-specific type II C-type lectin, mediates antigen capture and is a potent inhibitor of interferon alpha/beta induction. J. Exp. Med. 194:1823-1834.
25. Sadhu, C., B. Masinovsky, K. Dick, C. G. Sowell, and D. E. Staunton. 2003. Essential role of phosphoinositide 3-kinase delta in neutrophil directional movement. J. Immunol. 170:2647-2654.
26. Camps, M., T. Ruckle, H. Ji, V. Ardissone, F. Rintelen, J. Shaw, C. Ferrandi, C. Chabert, C. Gillieron, B. Francon, T. Martin, D. Gretener, D. Perrin, D. Leroy, P. A. Vitte, E. Hirsch, M. P. Wymann, R. Cirillo, M. K. Schwarz, and C. Rommel. 2005. Blockade of PI3 Kgamma suppresses joint inflammation and damage in mouse models of rheumatoid arthritis. Nat. Med. 11:936-943.
27. Gillooly, D. J., A. Simonsen, and H. Stenmark. 2001. Phosphoinositides and phagocytosis. J. Cell Biol. 155:15-17.
28. Vieira, O. V., R. J. Botelho, L. Rameh, S. M. Brachmann, T. Matsuo, H. W. Davidson, A. Schreiber, J. M. Backer, L. C. Cantley, and S. Grinstein. 2001. Distinct roles of class I and class III phosphatidylinositol 3-kinases in phagosome formation and maturation. J. Cell Biol. 155:19-25.
29. Ishii, K. J., F. Takeshita, I. Gursel, M. Gursel, J. Conover, A. Nussenzweig, and D. M. Klinman. 2002. Potential role of phosphatidylinositol 3 kinase, rather than DNA-dependent protein kinase, in CpG DNA-induced immune activation. J. Exp. Med. 196:269-274.
30. Dai, J., N. J. Megjugorac, S. B. Amrute, and P. Fitzgerald-Bocarsly. 2004. Regulation of IFN regulatory factor-7 and IFN-alpha production by enveloped virus and lipopolysaccharide in human plasmacytoid dendritic cells. J. Immunol. 173:1535-1548.
31. O'Keeffe, M., R. J. Grumont, H. Hochrein, M. Fuchsberger, R. Gugasyan, D. Vremec,
K. Shortman, and S. Gerondakis. 2005. Distinct roles for the NF-kappaBl and c-Rel transcription factors in the differentiation and survival of plasmacytoid and conventional dendritic cells activated by TLR-9 signals. Blood 106:3457-3464.
32. Yang, C. H., A. Murti, S. R. Pfeffer, J. G. Kim, D. B. Donner, and L. M. Pfeffer. 2001. Interferon alpha/beta promotes cell survival by activating nuclear factor kappa B through phosphatidylinositol 3-kinase and Akt. J. Biol. Chem. 276:13756-13761.
33. Kawai, T., S. Sato, K. J. Ishii, C. Coban, H. Hemmi, M. Yamamoto, K. Terai, M. Matsuda, J. Inoue, S. Uematsu, O. Takeuchi, and S. Akira. 2004. Interferon-alpha induction through Toll-like receptors involves a direct interaction of IRF7 with MyD88 and TRAF6. Nat. Immunol. 5:1061-1068.
34. Shinohara, M. L., L. Lu, J. Bu, M. B. Wemeck, K. S. Kobayashi, L. H. Glimcher, and H. Cantor. 2006. Osteopontin expression is essential for interferon-alpha production by plasmacytoid dendritic cells. Nat. Immunol. 7:498-506.
35. Duramad, O., K. L. Fearon, B. Chang, J. H. Chan, J. Gregorio, R. L. Coffman, and F. J. Barrat. 2005. Inhibitors of TLR-9 act on multiple cell subsets in mouse and man in vitro and prevent death in vivo from systemic inflammation. J. Immunol. 174:5193-5200.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention.

What is claimed is:

1. A method of inhibiting type I IFN production in a cell, the method comprising the step of administering to the cell a composition comprising a PI3K subunit inhibitor specific for the delta subunit of PI3K.

2. The method of claim 1, wherein the cell is a human plasmacytoid dendritic cell pDC).

3. The method of claim 1, wherein the composition further comprises a pharmaceutically acceptable excipient.

4. The method of claim 1, wherein the composition comprising the PI3K inhibitor specific for the delta subunit of PI3K regulates IRF-7 nuclear transport

5. The method of claim 1, wherein the composition comprising the PI3K inhibitor specific for the delta subunit of PI3K suppresses a Toll-like receptor (TLR).

6. The method of claim 5, wherein the TLR is selected from the group consisting of TLR9, TLR7/8 and/or a TLR7/8/9.

7. A method of treating, preventing or delaying the onset of a disease caused or characterized by the presence of pathogenic type I IFN, the method comprising the step of inhibiting type I IFN according to the method of claim 1.

8. The method of claim 7, wherein the disease is an autoimmune disease.

9. The method of claim 8, wherein the autoimmune disease is systemic lupus erythematosus (SLE), rheumatoid arthritis, psoriasis or Sjögren's disease.

10. A kit for carrying out the method of claim 1, the kit comprising a PI3K delta subunit inhibitor in a suitable container.

11. The kit of claim 10, further comprising instructions for use of the inhibitor in regulation of type I IFN.
